(19) United States
(12) Patent Application Publication
(10) Pub. No.: US 2026/0269078 A1
(43) Pub. Date: Sep. 10, 2026

(54) METHOD AND APPARATUS FOR CALCULATING RARE VARIANT POLYGENIC RISK SCORE

(71) Applicant: SEOUL NATIONAL UNIVERSITY R&DB FOUNDATION, Seoul (KR)

(72) Inventors: Seunggeun LEE, Seoul (KR); Kisung NAM, Seoul (KR)

(21) Appl. No.: 19/665,410

(22) Filed: May 1, 2026

Related U.S. Application Data
(63) Continuation of application No. PCT/KR2024/017238, filed on Nov. 5, 2024.

(30) Foreign Application Priority Data
Nov. 6, 2023 (KR) 10-2023-0151373
Nov. 5, 2024 (KR) 10-2024-0154841

(51) Int. Cl. G16H 50/30

(52) U.S. Cl. CPC G16H 50/30

(57) ABSTRACT

An embodiment relates to a method for calculating a polygenic risk score. The method includes calculating an estimated value of a phenotype for genome data of an individual using a preset first algorithm, classifying rare genetic variants for each gene included in the genome data into a plurality of groups according to a specific criterion, estimating an effect size of the rare genetic variants using a preset second algorithm based on a genotype matrix for each of the plurality of groups and the estimated value of the phenotype, and calculating a rare variant polygenic risk score using a preset third algorithm based on the genotype matrix for each of the plurality of groups and the effect size of the rare genetic variants.

### CROSS-REFERENCE TO RELATED APPLICATION

This application is based on and claims priority under 35 U.S.C. § 119 to Korean Patent Application No. 10-2023-0151373, filed on Nov. 6, 2023, No. 10-2024-0154841, filed on Nov. 5, 2024 in the Korean Intellectual Property Office, and is a continuation of International Application No. PCT/KR2024/017238 filed on Nov. 5, 2024, the disclosure of which is incorporated by reference herein in its entirety.

### BACKGROUND

### Technical Field

The present invention relates to a method and an apparatus for calculating a rare variant polygenic risk score, and more particularly, to a method and an apparatus for calculating an effect size of a rare genetic variant and calculating a rare variant polygenic risk score based on the effect size of the rare genetic variant.

### Background Art

Genetic variants may be classified into common genetic variants and rare genetic variants according to the frequency of occurrence within a population.

Because it is difficult to correctly estimate an effect of rare genetic variants on diseases using a conventional genome-wide association study (Genome-wide association study; GWAS), current genome-based disease prediction models use only information on common genetic variants. However, rare genetic variants are known to have a much larger effect size on diseases than

common genetic variants, and functionally often have a significant influence on diseases. Accordingly, estimation of the effect size of rare genetic variants is essential for improving performance of disease prediction models.

As methods for analyzing association of rare genetic variants, Burden test, SKAT, and SKAT-O are widely used.

In the case of the Burden test, the Burden test is a method capable of statistically testing measurement of an effect size and presence or absence of association under an assumption that all genetic variants within the same gene have the same effect size. However, because the basic assumption is very far from reality, there is a problem in that it is difficult to regard the Burden test as accurately representing the effect size of each genetic variant.

In addition, methods such as SKAT and SKAT-O relax such assumptions and allow testing of presence or absence of association under assumptions closer to reality. However, there exists a limitation in that such methods cannot measure the effect size.

Accordingly, there is a need for a method capable of calculating a polygenic risk score for rare genetic variants by more accurately measuring the effect size of rare genetic variants under realistic assumptions.

### SUMMARY

### Problem to be Solved

The present invention is intended to solve the problems of the above-described conventional technology, and an object of the present invention is to provide a method and an apparatus for calculating an effect size of rare genetic variants and calculating a rare variant polygenic risk score based on the effect size of the rare genetic variants.

The technical problems to be achieved by the present invention are not limited to the above-described technical problems, and other technical problems of the present invention may be derived from the following description.

### Means for Solving the Problem

As a technical means for solving the above-described technical problem, an embodiment according to a first aspect of the present disclosure provides a method for calculating a polygenic risk score. The method comprises: calculating an estimated value of a phenotype for genome data of an individual using a preset first algorithm; classifying rare genetic variants for each gene included in the genome data into a plurality of groups according to a specific criterion, and estimating an effect size of the rare genetic variants using a preset second algorithm based on a genotype matrix for each of the plurality of groups and the estimated value of the phenotype; and calculating the rare variant polygenic risk score using a preset third algorithm based on the genotype matrix for each of the plurality of groups and the effect size of the rare genetic variants.

As a technical means for solving the above-described technical problem, an embodiment according to a second aspect of the present disclosure provides a polygenic risk score calculating apparatus. The apparatus comprises: a communication module, at least one processor, and a memory electrically connected to the processor and storing at least one code executed in the processor. The memory stores code that, when executed through the processor, causes the processor to calculate an estimated value of a phenotype for genome data of an individual using a preset first algorithm, classify rare genetic variants for each gene included in the genome data into a plurality of groups according to a specific criterion, estimate an effect size of the rare genetic variants using a preset second algorithm based on a genotype matrix for each of the plurality of groups and the estimated value of the phenotype, and calculate the rare variant polygenic risk score using a preset third algorithm based on the genotype matrix for each of the plurality of groups and the effect size of the rare genetic variants.

### Effects of the Invention

According to the present invention, the method excludes an extreme assumption used in a Burden test-based method conventionally used for estimating an effect size of rare genetic variants, thereby reflecting actual biological characteristics.

In addition, according to the present invention, the effect size may be stably estimated using an empirical Bayes method, which is a shrinkage method.

In addition, according to the present invention, for binary variables, the effect size may be corrected by additionally performing Firth bias correction.

In addition, according to the present invention, by using singular value decomposition used in FaST-LMM, the effect size may be analyzed within a short time even for large-scale genome data.

The effects of the present invention are not limited to the above-described effects and include all effects understood from the following description.

### BRIEF DESCRIPTION OF THE DRAWINGS

FIG. 1 is a diagram illustrating a server and a terminal communicatively connected thereto according to an embodiment of the present invention.

FIG. 2 is a diagram illustrating a detailed configuration of the server illustrated in FIG. 1.

FIGS. 3 and 4 are diagrams illustrating an example of a form of genome data.

FIGS. 5 to 7 are diagrams illustrating an example of measuring an effect size of rare genetic variants for Type 2 Diabetes.

FIG. 8 is a flowchart illustrating a sequence of a method for estimating an effect size of rare genetic variants according to another embodiment of the present invention.

FIG. 9 is a flowchart illustrating a sequence of detailed steps of the method for estimating the effect size of rare genetic variants illustrated in FIG. 8.

### DETAILED DESCRIPTION

Hereinafter, the present disclosure will be described in detail with reference to the accompanying drawings. However, the present disclosure may be implemented in various different forms and is not limited to the embodiments described herein. In addition, the accompanying drawings are provided only to facilitate understanding of the embodiments disclosed in the present specification, and the technical spirit disclosed in the present specification is not limited by the accompanying drawings. All terms including technical terms and scientific terms used herein should be interpreted as having meanings generally understood by those skilled in the art to which the present disclosure belongs. Terms defined in advance should be interpreted as additionally having meanings consistent with related technical literature and the presently disclosed content, and unless otherwise defined, should not be interpreted as having overly ideal or overly restrictive meanings.

In the drawings, portions unrelated to the description are omitted in order to clearly describe the present disclosure, and sizes, forms, and shapes of respective components illustrated in the drawings may be variously modified. Throughout the specification, identical or similar reference numerals are assigned to identical or similar parts.

In the following description, suffixes such as “module” and “unit” for components are assigned or used interchangeably only for convenience in preparing the specification, and do not themselves have meanings or roles distinguished from each other. In addition, in describing the embodiments disclosed in the present specification, when it is determined that a detailed description of related known technology may obscure the gist of the embodiments disclosed in the present specification, the detailed description thereof is omitted.

Throughout the specification, when a portion is described as being “connected (coupled, contacted, or joined)” to another portion, this includes not only a case in which the portion is “directly connected (coupled, contacted, or joined)” but also a case in which the portion is “indirectly connected (coupled, contacted, or joined)” with another member interposed therebetween. In addition, when a portion is described as “including (having or provided with)” a certain component, this means that the portion may further include other components unless otherwise specifically stated, rather than excluding other components.

In the present specification, ordinal terms such as first and second are used only for the purpose of distinguishing one component from another component, and do not limit the order or relationship of the components. For example, a first component of the present disclosure may be referred to as a second component, and similarly, a second component may also be referred to as a first component. Forms of singular expressions used in the present specification should be interpreted as including plural expressions unless clearly indicated otherwise.

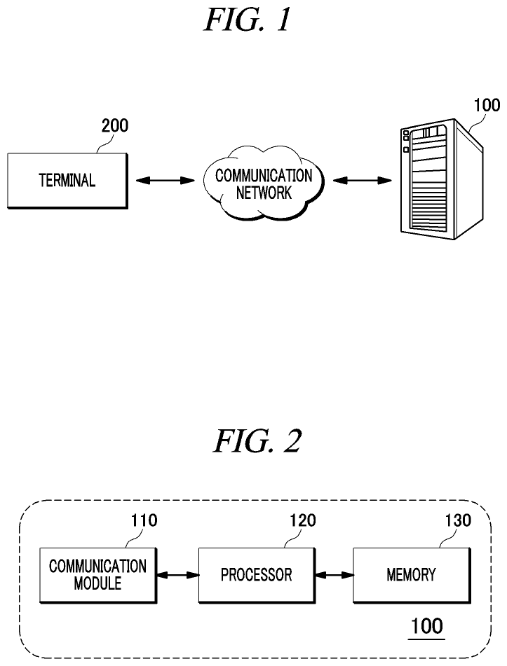

FIG. 1 is a diagram illustrating a server and a terminal communicatively connected thereto according to an embodiment of the present invention.

Referring to FIG. 1, the server (100) may be communicatively connected with the terminal (200) through a preset communication network.

The server (100) may be an apparatus for calculating a rare variant polygenic risk score.

The server (100) calculates an estimated value of a phenotype for genome data of an individual using a preset first algorithm.

The server (100) classifies rare genetic variants for each gene included in the genome data into a plurality of groups according to a specific criterion, and estimates an effect size of the rare genetic variants using a preset second algorithm based on a genotype matrix for each of the plurality of groups and the estimated value of the phenotype.

The server (100) calculates the rare variant polygenic risk score using a preset third algorithm based on the genotype matrix for each of the plurality of groups and the effect size of the rare genetic variants.

The terminal (200) may transmit genome data of an individual including at least one of whether the individual has been diagnosed with a disease, age, sex, and genetic factors to the server (100), and may receive the rare variant polygenic risk score from the server (100).

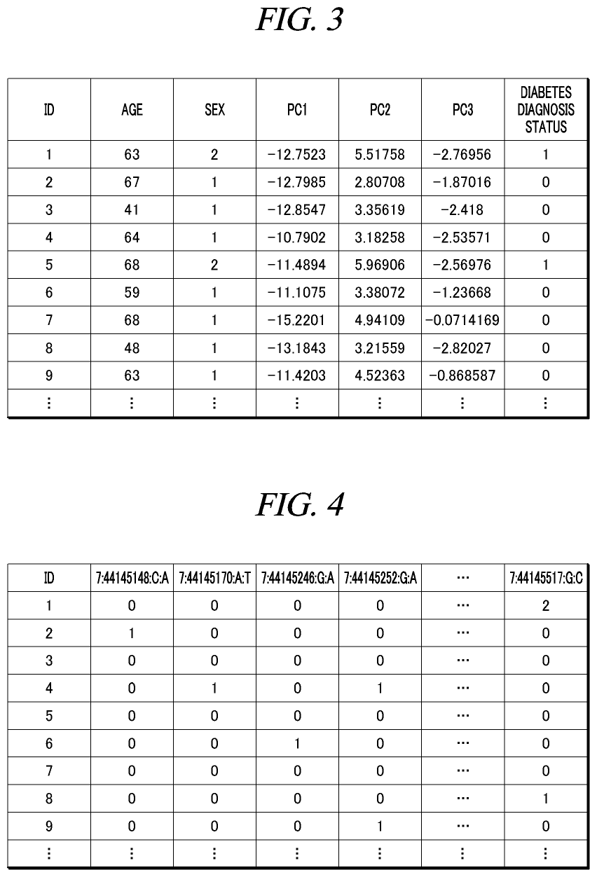

The terminal (200) may refer to any type of handheld-based wireless communication device such as a notebook, desktop, laptop equipped with a WEB browser, or a wireless communication device ensuring portability and mobility, or a smartphone or a tablet PC. FIG. 2 is a diagram illustrating a detailed configuration of the server illustrated in FIG. 1.

Referring to FIG. 2, the server (100) may include a communication module (110), a processor (120), and a memory (130).

The communication module (110) may include a device including hardware and software necessary for transmitting and receiving signals such as control signals or data signals through wired or wireless connection with another network device.

The communication module (110) may receive genome data of an individual including at least one of whether the individual has been diagnosed with a disease, age, sex, and genetic factors from the terminal, and may transmit the rare variant polygenic risk score to the terminal.

The processor (120) may include various types of devices for controlling and processing data. The processor (120) may refer to a data processing device embedded in hardware and having a physically structured circuit for performing functions represented by code or instructions included in a program.

In one example, the processor (120) may be implemented in the form of a microprocessor, a central processing unit (CPU), a processor core, a multiprocessor, an application-specific integrated circuit (ASIC), or a field programmable gate array (FPGA), but the scope of the present invention is not limited thereto.

The processor (120) performs operations according to code stored in the memory (130).

The memory (130) may store at least one of information and data input through the communication module (110), information and data necessary for functions performed by the processor (120), and data generated according to execution of the processor (120).

The memory (130) should be interpreted as collectively referring to a non-volatile storage device that continuously maintains stored information even when power is not supplied and a volatile storage device that requires power to maintain stored information. The memory (130) may include, in addition to a volatile storage device requiring power to maintain stored information, cloud storage, a solid-state drive (SSD), a magnetic storage media, or a flash storage media, but the scope of the present invention is not limited thereto.

The memory (130) is electrically connected to the processor (120) and stores at least one code executed in the processor (120). The memory (130) stores code that, when executed through the processor (120), causes the processor (120) to perform the following functions and procedures.

The memory (130) stores code that causes the processor (120) to calculate an estimated value of a phenotype for genome data of an individual using a preset first algorithm. For example, the memory (130) may store code that causes the processor (120) to calculate the estimated value of the phenotype using a generalized linear mixed model (Generalized Linear Mixed Model; GLMM).

Here, the genome data may include at least one of an individual identification number, age, sex, genetic information, and whether the individual has a disease.

The memory (130) may store code that causes the processor (120) to calculate the estimated value of the phenotype by considering covariates for at least one of age, sex, and genetic factors, genetic relationships, and errors.

The preset first algorithm may have different forms depending on whether the phenotype is a continuous variable or a binary variable, as shown in Equation 1.

$$y = Xa + b + ϵ ( when y is a continuous variable ) \qquad [ Equation 1 ]$$

$$logit ( P ( y = 1 ) ) = Xa + b + ϵ ( when y is a binary variable )$$

Here, y is a phenotype, X is a covariate including at least one of age, sex, and genetic factors, a is a coefficient, b is a random effect term following an MVN(0, σ_{g}K) distribution and represents a genetic relationship, and e is an error term following an MVN(0,σₑI) distribution.

For example, the continuous variable may be a variable that can be represented by continuous numerical values such as height, weight, and age, and the binary variable may be a variable having one of two possible answers such as sex and presence or absence of a disease.

The memory (130) stores code that causes the processor (120) to classify rare genetic variants for each gene included in the genome data into a plurality of groups according to a specific criterion, and estimate an effect size of the rare genetic variants using a preset second algorithm based on a genotype matrix for each of the plurality of groups and the estimated value of the phenotype.

The memory (130) may store code that causes the processor (120) to classify rare genetic variants into a plurality of groups according to a specific criterion and to configure a genotype matrix for each of the plurality of groups.

In one example, the memory (130) may store code that causes the processor (120) to classify rare genetic variants into a plurality of groups based on a risk level of the rare genetic variants. For example, the memory (130) may store code that causes the processor (120) to classify rare genetic variants into one of loss-of-function (Loss-of-function; LoF), missense variants (missense), and synonymous variants (synonymous), and to configure a genotype matrix. Here, the genotype matrix may be G_{LoF,j}, G_{mis,j} and G_{syn,j} respectively.

The memory (130) may store code that causes the processor (120) to classify genes as ultra-rare genetic variants when the number of alleles of the genes included in the genome data is less than a preset number, and to group the ultra-rare genetic variants into a single rare genetic variant. For example, ultra-rare variants having an allele count of fewer than 10 may be collapsed into a single indicator variable (or burden variable) representing the presence of one or more such variants within a gene.

The memory (130) may store code that causes the processor (120) to estimate an effect size of rare genetic variants using a second algorithm based on the genotype matrix and the estimated value of the phenotype.

The second algorithm may be Equation 2.

$$\tilde{y} = {G}_{LoF , j} {β}_{LoF , j} + ϵ \qquad [ Equation 2 ]$$

$$\tilde{y} = {G}_{mis , j} {β}_{mis , j} + ϵ$$

$$\tilde{y} = {G}_{syn , j} {β}_{syn , j} + ϵ$$

Here, for a continuous variable, \tilde{y}=y−ŷ and for a binary variable,

$$\tilde{y} = \frac{d {η}_{i}}{d {μ}_{i}} ( {y}_{i} - {μ}_{i} )$$

and β_{LoF,j}, β_{mis,j} and β_{syn,j} may each be a random effect term representing an effect size of a genetic variant.

Each of β_{LoF,j}, β_{mis,j} and β_{syn,j} may follow an MVN(0, τΣ) distribution. Here, Σ may be an arbitrary variance-covariance matrix, but in experiments performed in the present method, it is assumed to be an identity matrix I.

Accordingly, the effect size of the rare genetic variants may be estimated as shown in Equation 3.

$$E ( β \vert \tilde{Y} ) = τ \sum {( G \sum {G}^{T} + \frac{{σ}^{2}}{τ} I )}^{- 1} \tilde{Y} \qquad [ Equation 3 ]$$

Here, G is a genotype matrix (genotype matrix), G^{T} is a transpose matrix of the genotype matrix (transpose matrix), Σ is a variance-covariance matrix of a prior distribution of the effect size, τ is a variance component estimated through Equation 2, and σ² may represent a variance of \tilde{Y}.

The memory (130) may store code that causes the processor (120) to re-estimate the effect size based on a Firth bias correction method when the effect size is greater than or equal to a preset threshold.

The memory (130) may store code that causes the processor (120) to re-estimate the effect size through a Firth bias correction method to which an L2 penalty term is added as shown in Equation 4 when the effect size is greater than or equal to a preset threshold.

$$\hat{{β}_{*}} = arg max ( log L ( β ) + \frac{1}{2} log \vert I ( β ) \vert + λ {‖ β ‖}_{2} \qquad [ Equation 4 ]$$

The memory (130) stores code that causes the processor (120) to calculate a rare variant polygenic risk score (rare variant polygenic risk score; RVPRS) according to Equation 5 based on the effect size of the rare genetic variants.

$$RVPRS = {\sum }_{j} {\sum }_{f ∈ \{ LoF , mis , syn \}} {G}_{f , j} {β}_{f , j} \qquad [ Equation 5 ]$$

The memory (130) may store code that causes the processor (120) to calculate a final polygenic risk score by assigning preset weights to each of a rare variant polygenic risk score and a polygenic risk score (polygenic risk score; PRS) for common genetic variants, and to generate a disease prediction model for predicting a disease based on the final polygenic risk score.

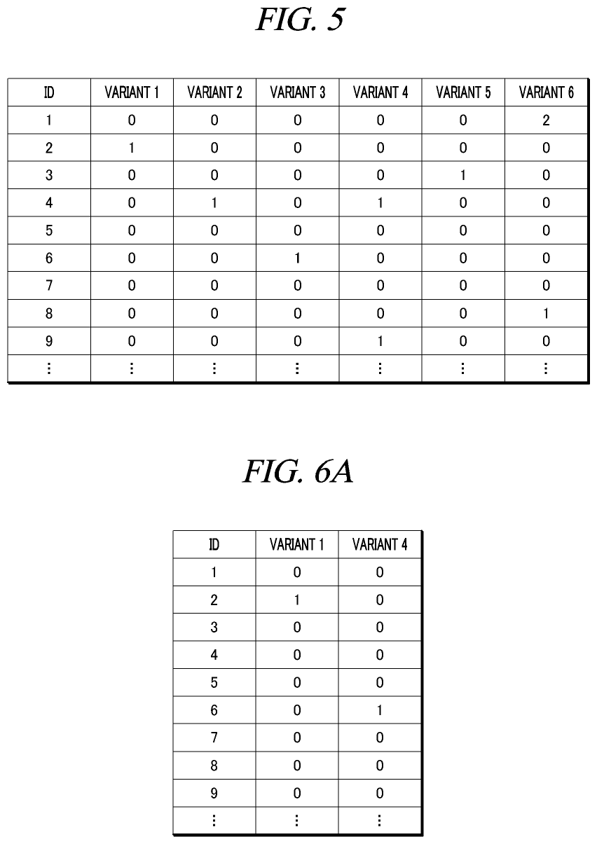

FIGS. 3 and 4 are diagrams illustrating an example of a form of genome data. More specifically, FIG. 3 is an example of a form of data corresponding to covariates, and FIG. 4 is an example of a form of data corresponding to genetic information.

Referring to FIGS. 3 and 4, the data corresponding to covariates may include information on whether each individual has been diagnosed with a disease, age, sex, and genetic principal components (PC). Here, genetic principal components capturing ancestry or population structure.

The data corresponding to genetic information may include information on each genetic variant. For example, each column of FIG. 4 may represent a genetic variant.

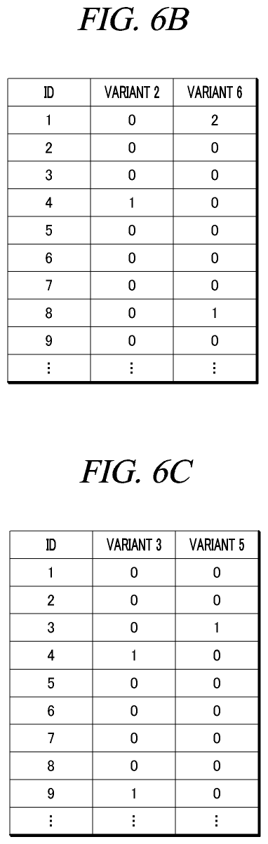

FIGS. 5 to 7 are diagrams illustrating an example of measuring an effect size of rare genetic variants for Type 2 Diabetes.

Referring to FIGS. 5 to 7, the polygenic risk score calculating apparatus may calculate an effect of covariates on a phenotype under a null model having only an intercept value without any input variables.

In this example, since a binary variable indicating whether a disease is diagnosed is handled, an effect of covariates on the phenotype may be calculated through a logistic mixed model as shown in Equation 6.

$$g ( x ) = ln ( \frac{x}{1 - x} ) \qquad [ Equation 6 ]$$

Here, g (x) may be a logit function.

For example, referring to FIGS. 3 and 4, a person with ID of 1 may have a vector of Yᵢ=1 and Xᵢ=(63,2, −12.7523, 5.51758, −2.76956) and K may be a matrix representing genetic relationships among individuals and may be an n & n matrix. Here, Yᵢ may represent whether the i-th person has been diagnosed with a disease, where 1 indicates that the person has the disease and 0 indicates that the person does not have the disease.

In general, a diagonal component represents a degree of relationship with oneself, where a close familial relationship may be represented as 1, and individuals who are not in a close familial relationship may be represented as 0. For example, when an i-th person and a j-th person are in a close familial relationship, a value representing a degree of the relationship between them may be reflected in coordinates (i, j) and (j, i) of K.

K may be expressed as shown in Equation 7.

$$K = [ \begin{matrix}1 & 0 & … & 0 \\ 0 & 1 & … & 0 \\ ⋮ & ⋮ & ⋱ & ⋮ \\ 0 & 0 & … & 1\end{matrix} ] \qquad [ Equation 7 ]$$

The polygenic risk score calculating apparatus may perform regression analysis using g, Yᵢ, Xᵢ, and K according to the first algorithm, and accordingly estimate a, bᵢ and

$${σ}_{g}^{2} .$$

The polygenic risk score calculating apparatus may calculate, for each individual, an estimated value of a phenotype μᵢ=P(y=1) using the estimated a, bᵢ and

$${σ}_{g}^{2} .$$

Here, the estimated value of the phenotype for each individual may be calculated by excluding covariates such as age, sex and genetic principal components. The estimated μᵢ may have different values and may have a form as shown in Equation 8.

$${μ}_{1} , {μ}_{2} , {μ}_{3} … {μ}_{n} = ( 0.088 , 0.174 , … , 0.1224 ) \qquad [ Equation 8 ]$$

The polygenic risk score calculating apparatus may calculate a working response

$$\text{?} = ( \frac{d {η}_{i}}{d {μ}_{i}} ) ( {y}_{i} - \text{?} )$$

using the estimated \hat{μ}_{ι}. The effect sizes of the genetic variants may then be estimated based on \tilde y and Equation 2.

The effect size of the genetic variant may have a form as shown in Equation 9.

$$\tilde{y} = ( \text{?} , \text{?} , … , \text{?} ) = ( 3.22 , - 0.45 , … , - 0.37 ) \qquad [ Equation 9 ]$$

Assuming that an effect size of rare genetic variants for a specific gene j is estimated within genome data, the polygenic risk score calculating apparatus may classify the rare genetic variants into a plurality of groups according to a specific criterion. For example, the genetic variants may be classified into at least one of loss-of-function, missense, and synonymous groups.

As shown in FIG. 5, in the genotype matrix, when it is assumed that variant 1 and variant 4 are loss-of-function, variant 2 and variant 6 are missense, and variant 3 and variant 5 are synonymous, G_{LoF,j}, G_{mis,j} and G_{syn,j} may be configured as shown in FIG. 6A, FIG. 6B, and FIG. 6C, respectively.

The polygenic risk score calculating apparatus may estimate the effect size of each variant according to Equation 2 based on y in Equation 9 and G_{LoF,j}, G_{mis,j} and G_{syn,j}.

For convenience of description, in Equation 2, it is assumed that β_{LoF,j}, β_{mis,j} and β_{syn,j} follow a multivariate normal distribution having a mean of 0 and variances of τ_{LoF,j}Σ_{LoF,j}, τ_{mis,j}, Σ_{mis,j} and τ_{syn,j}Σ_{syn,j} respectively, as prior distributions, and Σ may be an arbitrary variance-covariance matrix, but for convenience of description, it is assumed to be an identity matrix I.

The polygenic risk score calculating apparatus may estimate τ and σ by performing regression analysis for the three groups. The polygenic risk score calculating apparatus may estimate an effect size of each genetic variant by substituting the estimated τ and σ into Equation 3.

The effect size of each genetic variant may be as shown in FIG. 7A.

The polygenic risk score calculating apparatus may re-estimate and replace the effect size through Firth bias correction when an absolute value of the effect size exceeds a specific threshold.

The polygenic risk score calculating apparatus uses ln 2≈0.69 as the threshold, and in the case of variant 4 shown in FIG. 7A, since the effect size is greater than or equal to the threshold, the polygenic risk score calculating apparatus may re-estimate and update the effect size for variant 4.

The effect size of each genetic variant reflecting the re-estimated effect size may be as shown in FIG. 7B.

The polygenic risk score calculating apparatus may calculate a rare variant polygenic risk score (RVPRS) using the calculated effect sizes.

When a set of genes that are statistically significantly associated with a disease to be analyzed is denoted as J, the polygenic risk score may be calculated by calculating an RVPRS of an individual i for each gene j included in the set as shown in Equation 5 and summing the calculated values.

For example, when calculating an RVPRS of gene j for an individual with ID=1, a genotype may be (0, 0, 0, 0, 0, 2), and effect sizes of respective variants may be (−0.457,−0.137, 0.283, 1.382, 0.089,−0.096). Accordingly, RVPRS_i,j=0×(−0.457)+0×(−0.137)+0×(0.283)+0×(1.382)+0×(0.089)+2×(−0.096)=−0.192.

Assuming that there are 10 genes significantly associated with diabetes, the polygenic risk score calculating apparatus may calculate an RVPRS for each gene and then calculate a final value by summing the RVPRSs for the 10 genes.

The polygenic risk score calculating apparatus may construct a disease prediction model by integrating the RVPRS and a PRS calculated using common variants.

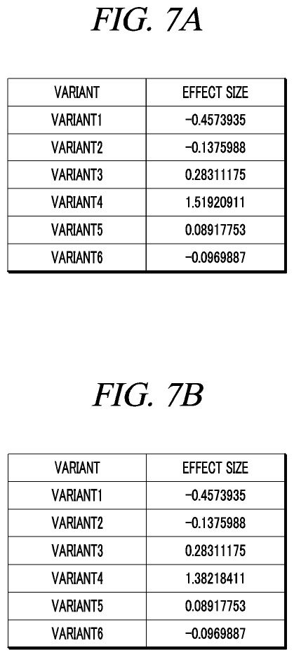

FIG. 8 is a flowchart illustrating a sequence of a method for estimating an effect size of rare genetic variants according to another embodiment of the present invention.

The method for estimating the effect size of rare genetic variants to be described below may be performed by the method for estimating the effect size of rare genetic variants or by the server (100) described above with reference to FIGS. 1 to 7. Accordingly, contents of the embodiment of the present disclosure described above with reference to FIGS. 1 to 7 may be equally applied to the embodiment to be described below, and redundant descriptions will be omitted. The steps described below are not necessarily performed in sequence, and an order of the steps may be variously set, and the steps may be performed substantially simultaneously.

Referring to FIG. 8, the method for estimating the effect size of rare genetic variants includes a step (S100) of calculating an estimated value of a phenotype, a step (S200) of estimating an effect size of rare genetic variants, and a step (S300) of calculating a rare variant polygenic risk score.

The step (S100) of calculating the estimated value of the phenotype is a step of calculating an estimated value of a phenotype for genome data of an individual using a preset first algorithm. The step (S100) of calculating the estimated value of the phenotype may include a step of

calculating the estimated value of the phenotype by considering covariates including at least one of age, sex, and genetic factors, genetic relationships, and errors.

The step (S200) of estimating the effect size of rare genetic variants is a step of classifying rare genetic variants for each gene included in the genome data into a plurality of groups according to a specific criterion, and estimating an effect size of the rare genetic variants using a preset second algorithm based on a genotype matrix for each of the plurality of groups and the estimated value of the phenotype. Here, the specific criterion may be set based on a risk level of the rare genetic variants.

The step (S300) of calculating the rare variant polygenic risk score may include a step of calculating the rare variant polygenic risk score using a preset third algorithm based on the genotype matrix for each of the plurality of groups and the effect size of the rare genetic variants.

The method for estimating the effect size of rare genetic variants may further include a step of calculating a final polygenic risk score by assigning preset weights to each of the rare variant polygenic risk score and a polygenic risk score for common genetic variants, and generating a disease prediction model for predicting a disease based on the final polygenic risk score.

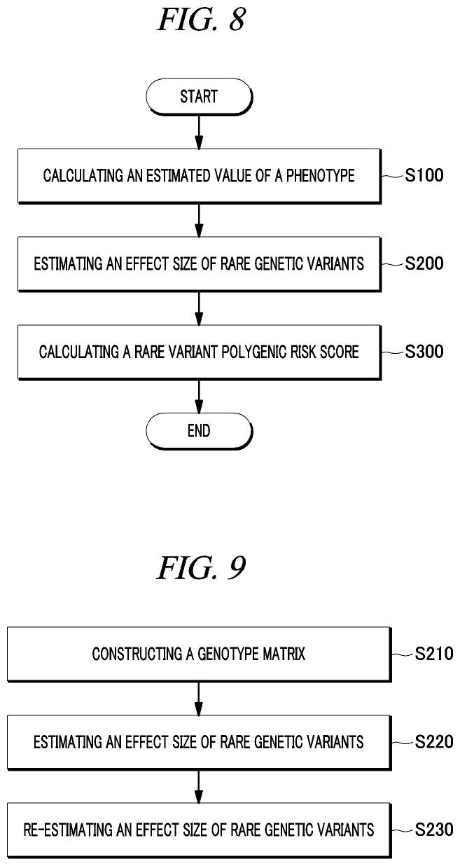

FIG. 9 is a flowchart illustrating a sequence of detailed steps of the method for estimating the effect size of rare genetic variants illustrated in FIG. 8.

Referring to FIG. 9, the step (S200) of estimating the effect size of rare genetic variants may include a step (S210) of constructing a genotype matrix, a step (S220) of estimating an effect size of rare genetic variants, and a step (S230) of re-estimating the effect size of rare genetic variants.

The step (S210) of constructing the genotype matrix may be a step of classifying rare genetic variants into a plurality of groups according to a specific criterion and constructing a genotype matrix for each of the plurality of groups.

The step (S210) of constructing the genotype matrix may include a step of classifying genes as ultra-rare genetic variants when the number of alleles of the genes included in the genome data is less than a preset number, and grouping the ultra-rare genetic variants into a single rare genetic variant.

The step (S220) of estimating the effect size of rare genetic variants may be a step of estimating the effect size of the rare genetic variants using the second algorithm based on the genotype matrix and the estimated value of the phenotype.

The step (S230) of re-estimating the effect size of rare genetic variants may be a step of re-estimating the effect size based on a Firth bias correction method when the effect size is greater than or equal to a preset threshold.

Those skilled in the art to which the present disclosure pertains will understand that various modifications may be easily made to other specific forms without departing from the technical spirit or essential characteristics of the present disclosure based on the above description. Therefore, the embodiments described above should be understood as illustrative in all respects and not restrictive. The scope of the present disclosure is defined by the appended claims, and all changes or modifications derived from the meaning and scope of the claims and equivalents thereof should be interpreted as being included in the scope of the present disclosure. The scope of the present application is defined by the appended claims rather than the above detailed description, and all changes or modifications derived from the meaning and scope of the claims and equivalents thereof should be interpreted as being included in the scope of the present application.

### MODE FOR CARRYING OUT THE INVENTION

The mode for carrying out the invention is substantially the same as the best mode for carrying out the invention described above.

### INDUSTRIAL APPLICABILITY

The present invention is applicable to disease prediction and thus has industrial applicability.

What is claimed is:

1. A method performed by a processor, the method comprising:
a) calculating an estimated value of a phenotype for genome data of an individual using a preset first algorithm;
b) classifying rare genetic variants for each gene included in the genome data into a plurality of groups according to a specific criterion, and estimating an effect size of the rare genetic variants using a preset second algorithm based on a genotype matrix for each of the plurality of groups and the estimated value of the phenotype; and
c) calculating a rare variant polygenic risk score using a preset third algorithm based on the genotype matrix for each of the plurality of groups and the effect size of the rare genetic variants.

2. The method of claim 1,
wherein the genome data includes at least one of an individual identification number, age, sex, genetic information, and whether the individual has a disease.

3. The method of claim 1,
wherein the specific criterion is set based on a risk level of the rare genetic variants.

4. The method of claim 1,
wherein the step a) includes calculating the estimated value of the phenotype by considering covariates including at least one of age, sex, and genetic factors, genetic relationships, and errors.

5. The method of claim 1,
wherein the first algorithm has a form of Equation 1 of y=Xa+b+ϵ when the phenotype is a continuous variable, and has a form of Equation 2 of logit(P(y=1))=Xa+b+ϵ when the phenotype is a binary variable, wherein y is the phenotype, X is a covariate, a is a coefficient, b is a term representing a genetic relationship, and ϵ is an error term.

6. The method of claim 1,
wherein the step b) includes:
b-1) classifying the rare genetic variants into the plurality of groups according to the specific criterion and constructing a genotype matrix for each of the plurality of groups;
b-2) estimating the effect size of the rare genetic variants using the second algorithm based on the genotype matrix and the estimated value of the phenotype; and
b-3) re-estimating the effect size based on a Firth bias correction method when the effect size is greater than or equal to a preset threshold.

7. The method of claim 6,
wherein the second algorithm has a form of Equation 3 of
$$\tilde{y} = {G}_{LoF , j} {β}_{LoF , j} + ϵ$$
$$\tilde{y} = {G}_{mis , j} {β}_{mis , j} + ϵ$$
$$\tilde{y} = {G}_{syn , j} {β}_{syn , j} + ϵ$$
wherein, for a continuous variable, \tilde{y}=y−ŷ and for a binary variable,
$$\tilde{y} = \frac{d {η}_{i}}{d {μ}_{i}} ( {y}_{i} - {μ}_{i} )$$
β_{LoF,j}, β_{mis,j} and β_{syn,j} are terms representing the effect sizes of the rare genetic variants, respectively, and e is an error term.

8. The method of claim 6,
wherein the step b-1) includes classifying genes as ultra-rare genetic variants when the number of alleles of the genes included in the genome data is less than a preset number, and grouping the ultra-rare genetic variants into a single rare genetic variant.

9. The method of claim 1,
wherein the third algorithm has a form of Equation 4 of RVPRS=ΣⱼΣ_{f∈{LoF,mis,syn}}G_{f,j}β_{f,j} wherein G_{f,j} is the genotype matrix, and β_{f,j} is the effect size of the rare genetic variants.

10. The method of claim 1, further comprising:
d) calculating a final polygenic risk score by assigning preset weights to each of the rare variant polygenic risk score and a polygenic risk score for common genetic variants, and generating a disease prediction model for predicting a disease based on the final polygenic risk score.

11. An apparatus for calculating a polygenic risk score, comprising:
a communication module;
at least one processor; and
a memory electrically connected to the processor and storing at least one code executed in the processor,
wherein the memory stores code that, when executed through the processor, causes the processor to calculate an estimated value of a phenotype for genome data of an individual using a preset first algorithm, classify rare genetic variants for each gene included in the genome data into a plurality of groups according to a specific criterion, estimate an effect size of the rare genetic variants using a preset second algorithm based on a genotype matrix for each of the plurality of groups and the estimated value of the phenotype, and calculate a rare variant polygenic risk score using a preset third algorithm based on the genotype matrix for each of the plurality of groups and the effect size of the rare genetic variants.

12. The apparatus of claim 11,
wherein the genome data includes at least one of an individual identification number, age, sex, genetic information, and whether the individual has a disease.

13. The apparatus of claim 11,
wherein the specific criterion is set based on a risk level of the rare genetic variants.

14. The apparatus of claim 11,
wherein the memory stores code that causes the processor to calculate the estimated value of the phenotype by considering covariates including at least one of age, sex, and genetic factors, genetic relationships, and errors.

15. The apparatus of claim 11,
wherein the first algorithm has a form of Equation 1 of y=Xa+b+ϵ when the phenotype is a continuous variable, and has a form of Equation 2 of logit(P(y=1))=Xa+b+ϵ when the phenotype is a binary variable, wherein y is the phenotype, X is a covariate, a is a coefficient, b is a term representing a genetic relationship, and e is an error term . . .

16. The apparatus of claim 11,
wherein the memory stores code that causes the processor to classify the rare genetic variants into the plurality of groups according to the specific criterion, construct a genotype matrix for each of the plurality of groups, estimate an effect size of the rare genetic variants using the second algorithm based on the genotype matrix and the estimated value of the phenotype, and re-estimate the effect size based on a Firth bias correction method when the effect size is greater than or equal to a preset threshold.

17. The apparatus of claim 16,
wherein the second algorithm has a form of Equation 3 of
$$\tilde{y} = {G}_{LoF , j} {β}_{LoF , j} + ϵ$$
$$\tilde{y} = {G}_{mis , j} {β}_{mis , j} + ϵ$$
$$\tilde{y} = {G}_{syn , j} {β}_{syn , j} + ϵ$$
wherein, for a continuous variable, \tilde{y}=y−ŷ and for a binary variable,
$$\tilde{y} = \frac{d {η}_{i}}{d {μ}_{i}} ( {y}_{i} - {μ}_{i} ) ,$$
β_{LoF,j}, β_{mis,j} and β_{syn,j} are terms representing the effect sizes of the rare genetic variants, respectively, and ϵ is an error term.

18. The apparatus of claim 16,
wherein the memory stores code that causes the processor to classify genes as ultra-rare genetic variants when the number of alleles of the genes included in the genome data is less than a preset number, and to group the ultra-rare genetic variants into a single rare genetic variant.

19. The apparatus of claim 11,
wherein the third algorithm has a form of Equation 4 of RVPRS=ΣⱼΣ_{f∈{LoF,mis,syn}}G_{f,j}β_{f,j} wherein G_{f,j} is the genotype matrix, and β_{f,j} is the effect size of the rare genetic variants.

20. The apparatus of claim 11,
wherein the memory stores code that causes the processor to calculate a final polygenic risk score by assigning preset weights to each of the rare variant polygenic risk score and a polygenic risk score for common genetic variants, and to generate a disease prediction model for predicting a disease based on the final polygenic risk score.
